(19) United States
(12) Patent Application Publication
(10) Pub. No.: US 2003/0126644 A1
(43) Pub. Date: Jul. 3, 2003

(54) Protocols for the generation of high yield, super productive transgenic plants disturbed in ran/ran-binding protein mediated cellular process

(76) Inventors: Soo-Hwan Kim, Taejeon-city (KR); Bin Goo Kang, Seol (KR); Woo Sung Lee, Suwon-city (KR); Ho-Il Kim, Kyonggi-do (KR); Hawk-Bin Kwon, Chungcheongnam-do (KR)

Correspondence Address: Rae Venter, Law Group, PO Box 1898, Monterey, CA, 93942-1898, US

(21) Appl. No.: 10/181,202

(22) PCT Filed: Aug. 28, 2001
(86) PCT No.: PCT/KR01/01450

(30) Foreign Application Priority Data
Aug. 28, 2000 (KR) 2000/50067
Aug. 27, 2001 (KR) 2001/51820

(51) Int. Cl. A01H 1/00; C07H 21/04; C12N 15/82; C12N 5/04

(52) U.S. Cl. 800/286; 435/320.1; 435/419; 536/23.6

(57) ABSTRACT

The present invention relates to methods for the generation of high yield, super-productive transgenic plants that are disturbed in Ran/Ran-binding proteins-mediated cellular processes. Specifically, the present intention relates to a method for generating transgenic plants overexpressing sense or antisense orientation of Ran or various Ran-binding proteins to modify the biological processes in which those proteins are involved. Those gene technologies provide ways to develop economically valuable super-productive crops whose biomass and yields are increased 1.5-2.5 times or more than those of currently available wild types. These increases in size or length can be seen in several organs of transgenic plants including leaf, stem, flower, roots, and seeds.

### BACKGROUND OF THE INVENTION

(a) Field of the Invention

The present invention describes a method for the generation of high yield, super-productive transgenic plants that are disturbed in Ran/Ran-binding proteins—mediated cellular processes. More particularly, the present invention relates to a method for generating transgenic plants overexpressing sense or antisense orientation of Ran or various Ran-binding proteins to modify the biological processes in which those proteins are involved. These gene technologies provide ways to develop economically valuable transgenic plants yielding super-productive crops resulting from an increase in their size or length, as can be seen in several organs of transgenic plants including leaf, stem, flower, roots, and seeds.

(b) Description of the Related Art

Ran (Ras-related nuclear) is a unique small GTP-binding protein predominantly localized in the nuclei. It plays an important role, with the aid of a family of Ran-binding proteins (RanBPs), in the regulation of protein nuclear transport and cell cycle progression.

Modulation of GTP- or GDP-bound state of Ran is achieved by the action of interacting (binding) proteins, such as RCC1 (a Ran nuclear guanine nucleotide exchange factor), Ran-GTPase-activating protein (RanGAP), Ran-binding protein 1 (RanBP1, a RanGAP cofactor), and Mog1 (a guanine nucleotide release factor) (Kahana, J. A., and Cleveland, D. W.,(1999) J. Cell Biol. 146, 1205-1210). RCC1 is a Guanine nucleotide exchange factor (GEF) that exchanges Ran-bound GDP with GTP, Ran-GAP is a Ran-GTPase activation protein that catalyzes the GTPase activity of Ran, and RanBP1 is a cofactor that aids the activity of Ran-GAP by stabilizing a Ran-GTP state (Hopper, A. K. Traglia, H. M., and Dunst, R. W. (1990) J. Cell Biol, 111, 309-321; Koepp, D. M., and Silver, P. A. (1996) Cell 87, 1-4). Such RanBPs cause the unequal distribution of Ran-GTP and Ran-GDP in cytoplasm and nucleoplasm in the prophase of cell division. Specifically, Ran-GTP becomes abundant on the nucleoplasmic side of a nuclear pore complex and Ran-GDP becomes predominant on the cytoplasmic side of the complex (Kahana, J. A., and Cleveland, D. W. (1999) J. Cell Biol. 146, 1205-1210).

Mitotic cells replicate, increase the number of chromosomes 2-fold, and transmit them to daughter cells evenly. This mitotic process consists of prophase, metaphase, anaphase and telophase. Ran/RanBP signal transduction pathway in concert with RanBP1, RanBPM, RCC1 and RanGAP, etc. have been recently shown to play critical roles in such processes in experiments using animal cell and yeast as a model organism. RCC1 maintains the concentration of Ran-GTP in nuclei at a high level while RanBP1 or RanGAP increases level of Ran-GDP in the same nuclei. RanBPM promotes the formations of chromosomal microtubule spindles in Ran-GTP dependent manner (Wilde, A., and Zheng, Y. (1999) Science 284, 1359-1362). These spindles move the chromosomes to opposite poles, thereby promote cell cycle progression and division (Nakamura, M., Masuda, H., Horri, J., Kuma, K. I., Yokoyama, N., Ohba, T., Nishitani, H., Miyata, T., Tanaka, M., and Nishimoto, T. (1998) J. Cell. Biol. 143, 1041-1052).

The mitotic effects of a RanBP1 in yeast have been reported in various studies (Battistoni, A., Guar guaglini, G., Degrassi, F., Pittoggi, C., Palena, A., Matteo, G. D., Pisano, C., Cundari, E., and Lavia, P. C. (1997) J. Cell Sci. 110, 2345-2357; He, X., Hayashi, N., Walcott, N. G., Azuma, Y., Patterson, T. E., Bischoff, F. R., Nishimoto, T., and Sazer, S. (1998) Genetics 148, 645-656; Kalab, P., Pu, R. T., and Dasso M. (1999) Curr. Biol. 9, 481-484). It is also reported that overexpression of AtRanBP1c in yeast inhibits formation of normal septum (Xia et al., 1996, Paint J. 10(4): 761-769); the nucleus is condensed and the arrangement of actin becomes irregular.

In addtion, Ouspenski reported that a RanBP1mutant (yrb1-21) caused defects in proper arrangement and the formation of mitotic spindles, and thus inhibited normal mitotic progression. (Ouspenski, I. I. (1998) Exp. Cell Res. 244, 171-183).

It was shown that plant Ran has similar function to that of yeast when the Ran was expressed in yeast. This implies that biological functions of Ran/RanBP pathways in plants might be similar to those of yeast or mammalian cells. Ach and Gruissem showed that a tomato Ran protein is functionally homologous to a yeast Ran-like protein (Ach, R. A. and Gruissem, W. (1994) Proc. Natl. Acad. Sci. USA 91, 5863-5867).

In addition, sequences of Ran1, Ran2 and Ran3 in Arabidopsis Thaliana and its binding proteins, AtRanBP1a and AtRanBP1b, have been identified recently. Their expression pattern and biochemical properties also have been reported (Haizel, T., Merkle, T., Pay, A., Fejes, E., and Nagy, F. (1997) Plant J. 11(1), 93-103). However, the function of those proteins in plants has never been studied so far.

### SUMMARY OF THE INVENTION

It is an object of the present invention to provide a method for generating transgenic plants by molecular genetic technologies exhibiting superior properties, namely having high yield and super-productivity.

It is another object of the present invention to provide recombinant vectors that can be transformed into crops by the above method.

It is another object of the present invention to provide genes that can be transformed into crops by the above method.

In order to achieve these objects, the present invention provides a Ran base sequence, as shown in SEQ. ID. No. 1.

In addition, the present invention also provides an AtRanBP1b base sequence, as shown in SEQ.ID.No. 2.

In addition, the present invention also provides an AtRanBP1c base sequence, as shown in SEQ.ID.No. 3.

In addition, the present invention also provides an antisense AtRanBP1b base sequence, as shown in SEQ.ID.No. 4, which suppresses the expression of endogenous AtRanBP1b gene.

In addition, the present invention also provides an antisense AtRanBP1c, as shown in SEQ.ID.No. 5, which suppresses the expression of endogenous AtRanBP1c gene.

The present invention also provides a recombinant vector harboring the antisense AtRanBP1b base sequence shown in SEQ.ID.No. 4 or a part of the antisense AtRanBP1b base sequence.

The present invention also provides a recombinant vector harboring the antisense AtRanBP1c base sequence shown in SEQ.ID.No. 5 or a part of the antisense AtRanBP1c base sequence.

The present invention also provides a recombinant vector harboring a gene which is selected from a group of Ran and Ran-binding proteins consisting of Ran, AtRanBP1a, AtRanBP1b, AtRanBP1c, RanGAP, RanBPM, RCC1 and RanBP1.

The present invention also provides transgenic plants transformed with a recombinant vector harboring a sense or an antisense base sequence of genes involved in Ran-mediated cellular processes.

The present invention also provides a method for generating transgenic plants transformed with a recombinant vector which contains a sense or an antisense base sequence of genes involved in Ran-mediated cellular processes, or knocked in the genes by T-DNA or transposons.

### BRIEF DESCRIPTION OF THE DRAWINGS

FIG. 1 shows a schematic diagram for the construct of a pLBJ21/PsRan vector.

FIG. 2 shows a schematic diagram for the construct of a pLBJ21/AtRanBP1b vector.

FIG. 3 shows a schematic diagram for the construct of a pLBJ21/AtRanBP1c vector.

FIG. 4 shows a schematic diagram for the construct of a pLBJ21/antisense AtRanBP1b vector.

FIG. 5 shows a schematic diagram for the construct of a pLBJ21/antisense AtRanBP1c vector.

FIG. 6 shows a genomic southern blot analysis for transgenic Arabidopsis (pLBJ21/PsRan).

FIG. 7 shows a genomic southern blot analysis for transgenic Arabidopsis (pLBJ21/antisense AtRanBP1c).

FIG. 8 shows a northern blot analysis for transgenic Arabidopsis (pLBJ21/Ps Ran).

FIG. 9 shows a northern blot of for transgenic Arabidopsis (pLBJ21/antisense AtRanBP1c).

FIG. 10 compares the root length of transgenic Arabidopsis (pLBJ21/PsRan) of the present invention and that of wild type plants.

FIG. 11 compares the length of roots for transgenic Arabidopsis plants (pLBJ21/antisense AtRanBP1c) of the present invention and that of wild type plants.

FIG. 12 compares the size of seeds for a transgenic Arabidopsis (pLBJ21/antisense AtRanBP1b) of the present invention and that of wild type plants.

FIG. 13 compares the size of flower for a transgenic Arabidopsis (pLBJ21/antisense AtRanBP1b) of the present invention and that of wild type plants.

FIG. 14 compares the size of leaves for a transgenic Arabidopsis (pLBJ21/antisense AtRanBP1b) of the present invention and that of wild type plants.

FIG. 15 compares adult the size of an adult plant for a pLBJ21/antisense AtRanBP1b transgenic plant of the present invention and that of wild type plants.

FIG. 16 compares the size of an adult plant for a transgenic tomato (pLBJ21/AtRanBP1b) of the present invention and that of wild type plants.

FIG. 17 compares the size of leaf for a transgenic tomato (pLBJ21/AtRanBP1b) of the present invention and that of wild type plants.

FIG. 18 compares the size of branch for a transgenic tomato (pLBJ21/AtRanBP1b) of the present invention and that of wild type plants.

FIG. 19 compares the size of immature fruit for a transgenic tomato (pLBJ21/AtRanBP1b) of the present invention and that of wild type plants.

### DETAILED DESCRIPTION AND THE INVENTION

The present invention will now be described in more detail.

The function of Ran or Ran-BP in plants has never been studied so far. The present inventor revealed that plant Ran and AtRanBP1c are very crucial in the regulation of auxin-induced cell cycle progression. The inventor developed transgenic plants changed in the levels of Ran or AtRanBP1c, and thus increased auxin sensitivity for mitotic root cells. This modification resulted in long-rooted transgenic plants. This inventor also discovered that AtRanBP1b is an important regulator of meiotic and mitotic progression, and revealed that AtRanBP1b controls cell cycle progression from metaphase to interphase in dividing plant cells. Transgenic plants changed in the level of AtRanBP1b have been generated by this invention, and these plants have been shown to carry a big plant phenotype and produce a significantly high yielded product. The big plant phenotype is defined later in this document.

The present invention established methods for generating transgenic plants of super-productive, high-yielded crops by overexpression or suppression of sense or antisense sequences for a group of proteins that are involved in Ran-mediated cellular processes.

Proteins involved in Ran-mediated cellular processes and targeted for the present invention are Ran (Haizel et al., 1997, Plant J 11 (1) 193-103) and Ran-binding proteins. It is desirable that Ran-binding proteins are preferably selected from the group of proteins consisting of AtRanBP1a (Haizel et al., 1997, Plant J 11(1): 93-103), AtRanBP1b (Haizel et al., 1997, Plant J 11(1): 93-103), AtRanBP1c (AT5 in Xia et al., 1996, Plant J. 10(4):761-769), RanGAP (Merril et al., 1999 Science, 283: 1742-1745), RanBPM (Nakamura et al., 1998, J. Cell. Biol., 143(4): 1041-1052), RCC1 (Fruno et al., 1991, Genomic, 11(2):459-461), and RanBP1 (Ouspenski et al., 1995, J. Biol. Chem.., 270(5): 1975-1978). The AtRanBP1a, AtRanBP1b and AtRanBP1c are Ran-binding proteins found in Arabidopsis, and the RanGAP, RanBPM, RCC1 and RanBP1 are Ran-binding proteins found in animals or yeasts, etc. Amino acid sequences of Ran proteins are well conserved among organisms; it is 90% or more identical to each other in plants, and is 70% or more identical to that of yeasts (Merkle et al., 1994, Plant J. 6(4): 555-565). The DNA sequence of AtRanBP1 in plants is 80% or more identical to each other, and it is 60% or more identical to that of other organisms (Haizel et al., 1997, Plant J. 11(1): 93-103). On the other hand, the amino acid sequence of CST20, a RanBP1 of yeast, is approximately 50% identical to that of RanBP1 of mice (Ouspenski et al., 1995, JBC 270(5): 1975-1978). The amino acid sequence of RanBPM of mice is almost identical to that of human, and approximately 30% identical to that of yeasts (Nakamura et al., 1998, J. Cell. Biol., 143(4):1041-149). The amino acid sequence of RanGAP of Drosophilae is 34 to 36% identical to those of RanGAP in yeasts and mice (Merril et al., 1999, Science, 12:283-287). Thus, it is reasonable to say that Ran is a family of proteins that are 70% or more identical in the amino acid sequences, and AtRanBP1b or AtRanBP1c is a family of proteins that are 50% or more identical in the amino acid sequences. Accordingly, other RanBPs such as RanGAP, RCC1, and RanBPM is a group of proteins that are 35% or more identical in their amino acid sequences.

Many Ran-binding proteins have a stretch of amino acid sequences called a Ran-binding domain (RanBD) where Ran protein binds to (Beddow et al., 1995, PNAS, 92:3328-3332). This Ran-binding domain aids the hydrolysis of GTP by binding to Ran protein. (Novoa et al., 1999, Mol. Biol. Cell., 10: 2175-2190).

Therefore, a group of proteins involved in the Ran-mediated cellular processes comprise a group of protein that contains the Ran-binding domain.

In addition, the present invention provides methods generating transgenic plants with modified levels of Ran or Ran-binding proteins (RanBP) by overexprssing, inhibiting or knocking-out these genes. These transgenic plants can be generated by the overexpression of Ran or RanBP, by the suppression of the endogenous genes by the expression of complimentary antisense RNA, or by the suppression of endogenous genes by the expression of the corresponding double-stranded RNA (RNA interference) using various plant promoters. These plants can be also generated by knocking-out the genes using UV-treatment, EMS-treatment, or transposon. The antisense method is a method for inhibiting the translation of a mRNA of target gene by expressing the complementary strand (antisense ) to the target genes in the organism to produce the bond between mRNA of the target genes and antisense mRNA, thereby causing the mRNA of the target genes not to be translated into protein.

Tomato and Arabidopsis have been generally used as model plants in the field of plant molecular biology. In the present invention, transgenic tomatoes or Arabidopsis are generated by transformation of recombinant vectors overexpressing or suppressing Ran or Ran-binding proteins. These recombinant vectors include any gene-expressing promoters that could express sense or antisense sequence of Ran or Ran-binding proteins, or sense and antisense sequence of the protein genes in the same times.

To generate transgenic plants mentioned above, PsRan which is a Ran protein of Pissum Satinum and shown in SEQ.ID.No. 1, was cloned from an etiolated pea plumule library and was inserted into a pLBJ21 vector to construct a recombinant vector pLBJ21/PsRan. This vector was deposited in the Korean collection for type cultures, as KCTC 0837BP. The pBJ21 vector is a pKYLX71 derivative plasmid in which a HindIII recognition site in the multi-cloning region of a pKYLX71 expression cassette is converted to an EcoRI recognition site (Schardl, C. L., Byrd, A. D., Benzion, G., Altschuler M. A., Hilderbrand, D. F., and Hunt, A. G. (1987) Gene 61, 1-11, 1987; Lloyd, A. M. N. Walbot, V., and Davis, R. W. (1992) Science 258,1773-1775). It is a binary vector, and it is used as a transformation vector in the present invention.

In addition, AtRanBP1b (shown in SEQ.ID.No. 2) and AtRanBP1c (shown in SEQ.ID.No. 3) were inserted into pLBJ21 vectors, and were deposited in the Korean collection for the type as pLBJ21/AtRanBP1b KCTC 0838BP and pLBJ21/AtRanBP1c KCTC 0839BP, respectively.

Three recombinant vectors pLBJ21/PsRan, pLBJ21/AtRanBP1b and pLBJ21/AtRanBP1c were transformed into Arabidopsis. Transgenic lines were selected to characterize transgenic phenotypes. Transgenic pLBJ21/PsRan (KCTC 0837BP) Arabidopsis showed a long root and big plant phenotype. Transgenic Arabidopsis transformed with pLBJ21/AtRanBP1b (KCTC 0838BP) showed a big plant phenotype. Transgenic Arabidopsis transformed with pLBJ21/AtRanBP1c (KCTC 0839BP) showed a long-root, increased seed weight, and big plant phenotype.

In addition, in order to generate transgenic plants suppressing genes involved in Ran-mediated cellular processes, antisense base sequences to AtRanBP1b and AtRanBP1c were respectively designed. The antisense AtRanBP1b base sequence was described in SEQ.ID.No. 4, and the antisense AtRanBP1c base sequence was described in SEQ.ID.No. 5, using a standard sequence listing software program. Transgenic plants whose Ran-mediated cellular processes are suppressed can be generated with recombinant vectors having a whole antisense sequences of SEQ.ID.No. 4 or SEQ.ID.No. 5, or a part of those base sequences. Nucleotide sequences should be 50 base pairs (bp) or more in length to be effective. In the case where the sense and antisense base sequences of genes are simultaneously expressed and suppress endogenous genes involved in Ran-mediated cellular processes in the transgenic plants, it is desirable to have the sequences preferably 26 bp or more in length (Parrish et al., 2000 Molecular Cell 6: 1077-1087; Chuang et al., Proc. Natl. Acad. Sci, USA 97:4985-4990).

In the present invention, antisense AtRanBP1b or AtranBP1c were inserted into pLBJ21 vectors to construct two kinds of recombinant vectors. The constructed recombinant vector, pLBJ21/antisense AtRanBP1b, was deposited under KCTC 0850BP, and the recombinant vector pLBJ21/antisense AtRanBP1c was deposited under KCTC 0851BP.

The two kinds of recombinant vectors, pLBJ21/antisense AtRanBP1b and pBLJ21/antisense AtRanBP1c, were transformed into Arabidopsis, and the phenotypes of tranformants were observed. As a result, transgenic Arabidopsis that was transformed with the recombinant vector pLBJ21/antisense AtRanBP1b (KCTC 0850BP) showed a big plant phenotype; virtually, sizes of all plant bodies are increased. For example, the leaf area, stem thickness, the volume and weight of the seed, height of plant, size of flower, length of trichome, and the number of seeds per plant were significantly increased. Transgenic Arabidopsis that was transformed with the recombinant vector PLBJ21/antisense AtRanBP1c (KOTC 0851BP) showed the phenotypes of long primary root growth, and the same big-plant as described above.

Sensitivity to auxin in the Arabidopsis plants transformed with the pLBJ21/antisense AtRanBP1c was tested. Auxin is a plant hormone that promotes germination of pollen, extension of the pollen tube, formation of lateral roots and flower buds, generation of sprouts or roots from callus, and the division and growth of the dividing cells. In the present invention, it was tested whether or not the sensitivity to auxin, that affects the division and growth of cells, is changed in the transformed plants. In general, a low concentration (10⁻⁶-10⁻⁷ M) of auxin in the roots promotes the division of root cells and subsequent root growth while inhibiting the growth of lateral roots, whereas a high concentration (10⁻⁶- 10⁻⁵ M) of auxin acts in the opposite manner.

Transgenic Arabidopsis transformed with pBUJ21/antisense AtRanBP1c of the present invention shows a hyper-sensitive response to auxin; even a low concentration of auxin such as pM supplied from the outside suppressed root growth and promoted the initiation of lateral root differentiation. In contrast in the wild-type plants, there was no response to the same concentration of auxin. In wild type plants, the lateral root differentiation was promoted at 10⁻⁷ M of auxin. Since the transgenic plants of the present invention have increased auxin-sensitivity, a very low concentration of endogenous auxin, that does not generally promote root growth in wild types, does in fact promote root growth in plants genetically modified by the present invention.

In addition, this invention generated tomatoes transformed with pBLJ21/antisense AtRanBP1b (KCTC0850BP), pBLJ21/antisense AtRanBP1c (KCTC0851BP), pBLJ21/PSRan (KCTC0837BP), pBLJ21/AtRanBP1b (KCTC0838BP), or pLBJ21/AtRanBP1c (KCTC0839BP).

Transgenic plants in which Ran-mediated cellular processes are modified showed the phenotypes of long root, big-sized seed, and big body. Thus, superior species can be developed by the method for generating transgenic plants modified in Ran-mediated cellular processes of the present invention as mentioned above.

The present invention will be explained in more detail with reference to the following Examples. However, the following Examples are to illustrate the present invention and the present invention is not limited to them.

### EXAMPLE 1

### Transgenic Plants Expressing PsRan Protein

(1) The Cloning of PsRan Gene

Using an EcoRI/XhoI gene segment of an ATTS1902 clone provided from Arabidopsis Cdna BANK (ARBC, Ohio) as a probe, a pea plumule cDNA expression library was screened to obtain PsRan genes.

(2) The Construction of a Recombinant Vector Containing PsRan Gene

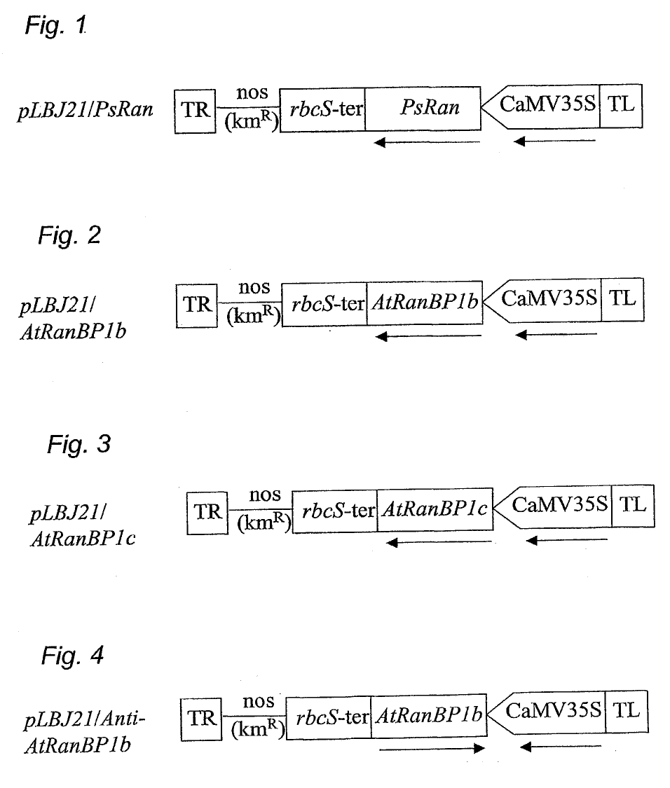

PsRan cDNA was cloned at EcoRI and XhoI sites in a pBluescript SK+ (Strategene, Calif.) vector. The genes were digested with the restriction enzymes EcoRI and XhoI, and then were cloned into a pLBJ21 vector that had been digested with the same restriction enzymes to construct a pLBJ21/PsRan expressing PsRan under a CaMV35S promoter, as shown in FIG. 1. The fusion vector was introduced into Agrobacterium tumefaciens GV3101 containing pMP90 plasmid by electroporation (Koncz, C., and Schell, J. (1986) Mol. Gen. Genet. 204, 383-396) to transform the recombinant vector into plants.

(3) The Generation of Arabidopsis Transformed with pLBJ21/PsRan

The pLBJ21/PsRan was introduced into root explants of Arabidopsis using Agrobacterium-mediated transformation method (Valvekens et al., D., Montagu, M. V., and Lijsebettens, m. v. (1998) Proc. Natl. Acad. Sci. USA 85, 5536-5540). T1 seeds from primary transformants were grown on a Germination Medium (Valvekens et al., D., Montagu, M. V., and Lijsebettens, M. V. (1998) Proc. Natl. Acad. Sci. USA 85, 5536-5540) and the transformants were selected with 50 μg/L of kanamycin. T2 seeds were recovered from the selected T1 plants, and were grown on the same medium to identify homozygous T2 seeds. Identified T2 seeds were used to observe the characteristics of the transgenic plants.

### EXAMPLE 2

### Transgenic Plants Expressing AtRanBP1b Protein

(1) The Cloning of AtRanBP1b Gene

A yeast two-hybrid screening method was used to clone Ran-binding proteins in Arabidopsis cDNA library using PsRan as a bait. Specifically, a full-length PsRan was amplified by the PCR method using a primer of SEQ.ID.No. 3 containing initial ATG codon and a primer of SEQ.ID.No. 4 containing stop codon. The amplified PsRan was digested with EcoRI and BamHI, and then was subcloned into a pGBT9 vector (Clonetech, Calif.) predigested with the same restriction enzymes. A recombinant pGBT9/PsRan vector was cotransformed into Saccharomyces cerevisiae Y190 together with an Arabidopsis cDNA library that was subcloned in pACT, and then a Ran binding protein, AtRanBP1b, was cloned according to the method described in Yeast Two Hybrid Screening published in Clonetech.

(2) The Construction of a Recombinant Vector, pLBJ21/AtRanBP1b, Containing AtRanBP1b Gene

A pLBJ21/AtRanBP1b recombinant vector was constructed by the same method as described in Example 1 except that AtRanBP1b was used instead of PsRan, as shown in FIG. 2. Specifically, a full length AtRanBP1b was PCR amplified using the primer of SEQ.ID.No. 8 comprising ATG and the primer of SEQ.ID.No. 9, and then the amplified genes were digested with XhoI/XbaI. The digested AtRanBP1b fragment was subcloned in pLBJ21 that was digested with the same enzymes to construct a recombinant vector pLBJ21/AtRanBP1b as shown in FIG. 2. The recombinant vector was introduced into Agrobacterium by electroporation as mentioned above.

(1) The Generation of Arabidopsis Transformed with pLBJ21/AtRanBP1b

Arabidopsis plants that are transformed with PJBL21/AtRanBP1b were generated by the same method as described in Example 1.

### EXAMPLE 3

### Transgenic Plants Expressing AtRanBP1c Gene

(1) The Cloning of AtRanBP1c Gene

AtRanBPlc was cloned by the yeast two-hybrid screening method as described in Example 2 (1).

(2) The Construction of a Recombinant Vector, pLBJ21/AtRanBP1c, Containing AtRanBP1c Gene

A pLBJ21/AtRanBP1c recombinant vector was constructed by the same method as described in Example 2 (2), except that AtRanBP1c was used instead of AtRanBP1b, as shown in FIG. 3. A full-length AtRanBP1c was PCR amplified using the primer of SEQ.ID.No. 10 and the primer of SEQ.ID.No. 11.

(3) The Generation of Arabidopsis Plants Transformed with pLBJ21/AtRanBP1c

Arabidopsis plants transformed with pLBJ21/AtRanBP1c were generated by the same method as described in Example 1.

### EXAMPLE 4

### Transgenic Plants Expressing Antisense AtRanBP1b

(1) The Preparation of an Antisense AtRanBP1b Base Sequence

A full length AtRanBP1b was amplified by RT-PCR from Arabidopsis total RNA, using primer 1 of SEQ.ID.No. 12 that has an XhoI enzyme recognition site in 3′-terminal cDNA of AtRanBP1b and primer 2 of SEQ.ID.No. 13 that has an XbaI recognition site in 5′-terminal cDNA

(2) The Construction of a Recombinant Vector, pLBJ21/antisense AtRanBP1b, Containing Antisense AtRanBP1b Base Sequence

The RT-PCR amplified AtRanBP1b was digested with XhoI/XbaI to prepare a DNA fragment having an antisense AtRanBP1b base sequence in the direction from XhoI to XbaI. Then, the DNA fragment was cloned into the region of pLBJ21 digested with XhoI/XbaI to construct a pLBJ21/antisense AtRanBP1b overexpressing an antisense AtRanBP1b under a CaMV35S promoter, as shown in FIG. 4. The pLBJ21/antisense AtRanBp1b was introduced into Agrobacterium tumefaciens GV3101 containing pMP90 plasmid by electroporation (Koncz, C., and Schell, J. (1986) Mol. Gen. Genet. 204, 383-396).

(3) The Generation of Transgenic Plants Transformed with pLBJ21/antisense AtRanBP1b

Transgenic plants transformed with PLBJ21/antisense AtRanBP1b were generated by the same method as described in Example 1.

### EXAMPLE 5

### Transgenic Plants Expressing Antisense AtRanBP1c

(1) The Preparation of an Antisense AtRanBP1c Base Sequence

A full length AtRanBP1c was RT-PCR amplified by the same method as described in Example 4 (1). Primers of SEQ.ID.No. 14 and SEQ.ID.No. 15 were used in RT-PCR.

(2) The Construction of a Recombinant Vector Containing an Antisense AtRanBP1c Base Sequence

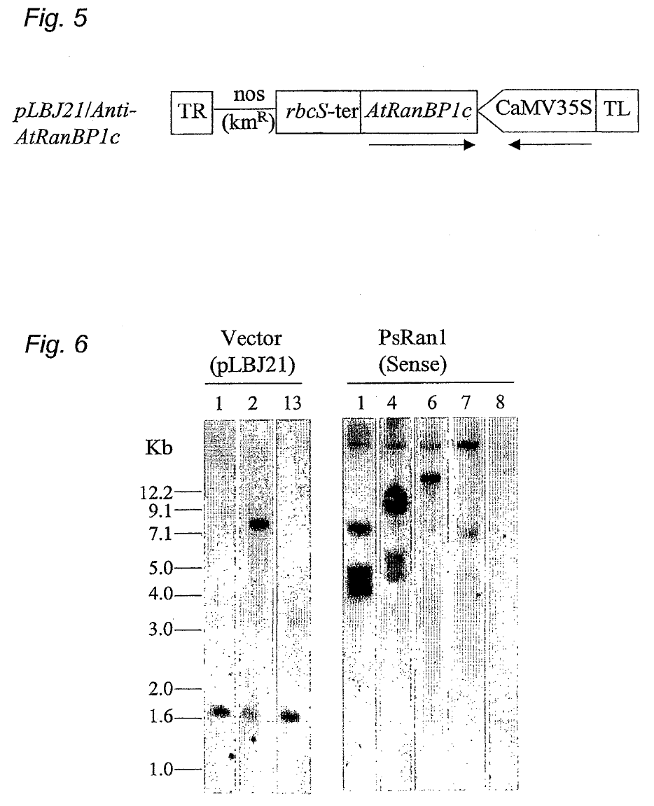

A pLBJ21/antisense AtRanBP1c recombinant vector was constructed by the same method as described in Example 4 except that AtRanBP1c was used instead of AtRanBP1 b, as shown in FIG. 5.

(3) The Generation of Arabidopsis Transformed with pLBJ21/Antisense AtRanBP1c

Arabidopsis plants transformed with pLBJ21/antisense AtRanBP1c were generated by the same method as described in Example 1.

### EXAMPLES 6 to 10

A pLBJ21/PsRan, pLBJ21/AtRanBP1b, pLBJ21/AtRanBP1c, pLBJ21/antisense AtRanBP1b, and pLBJ21/antisense AtRanBP1c recombinant vector were transformed into tomatoes by the same method as described in Examples 1 to 5 except a cotyledone of tomatoes was used instead of Arabidopsis root explants.

Experiment 1. The Identification of Transgenic Plants

(i) Genomic Southern Analysis

In order to identify whether or not the genes that were transformed into transgenic plants of the above Examples were stably introduced into the chromosomes of the plants, a Southern analysis was conducted with the genomic DNA of the transgenic plants.

Genomic DNA was isolated from 3 week-old plants, and it was digested with EcoRI. The digested DNA were separated on a 0.8% agarose gel by electrophoresis and transferred to a zeta-probe membrane (Biorad). The membrane was preincubated in 0.25 M sodium phosphate (PH 7.2) and 7% SDS at 65° C. for 30 minutes. [α-³²P]dATP-CaMV 35S promoter (a fragment of pB1221 vector digested with XbaI/HindIII) probe was added in the incubating solution and the membrane was further incubated at 65° C. for 20 hours. After the incubation, the membrane was washed with a solution containing 20 mM sodium phosphate, pH 7.2, and 5% SDS, and finally with the same solution containing 1% SDS at 65° C., for 1 hour. Then the membrane was exposed to a film to detect the inserted tansgene. As a result, transformed plants carrying CaMV35S promoter and the transgene were identified.

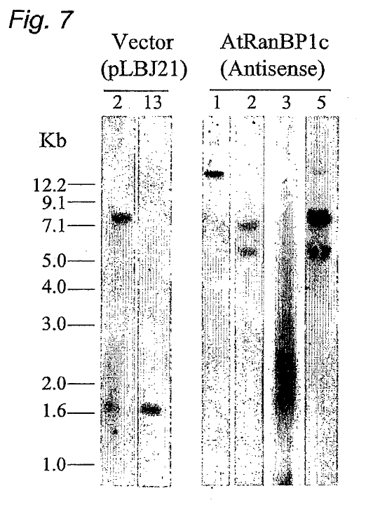

FIGS. 6 and 7 shows the Southern blotting analysis of the two different transgenic lines.

FIG. 6 shows a photograph of the DNA gel blot analysis (Southern blot analysis) of Arabidopsis transformed with pLBJ21/PsRan, and FIG. 7 shows a photograph of the DNA gel blot analysis of Arabidopsis transformed with pLBJ21/antsense AtRanBP1c. As shown in FIGS. 6 and 7, we generated many pLBJ21/PsRan and pLBJ21/antisense AtRanBP1c transgenic plants whose transgenes were inserted into the different chromosomes. (For example, CaMV35S fragments were identified by different bands with different length in the genome of Sense PsRan-1, -4, -6, -7, -8 plants).

(ii) Northern Blotting Analysis of RNA

In order to identify whether or not the transgenic plants, in which the introduction of genes was confirmed as shown above, express the introduced genes as well, they were further subjected to a Northern blotting analysis.

To do this, RNA of transgenic plants were extracted using a Trizol reagent (Gibco BRL) solution and separated on a 1.2% agarose gel containing 6% formaldehyde by electrophoresis. Northern blotting analysis was then conducted using the sense or antisense riboprobe of PsRan, AtRanBP1b or AtRanBP1c. The probe was prepared using a transcription kit, MAXIscript T3/T7 (Ambion, Tex.). After Northern hybridization, RNA was exposed to a intensifying screen and developed to measure the expression of corresponding RNAs.

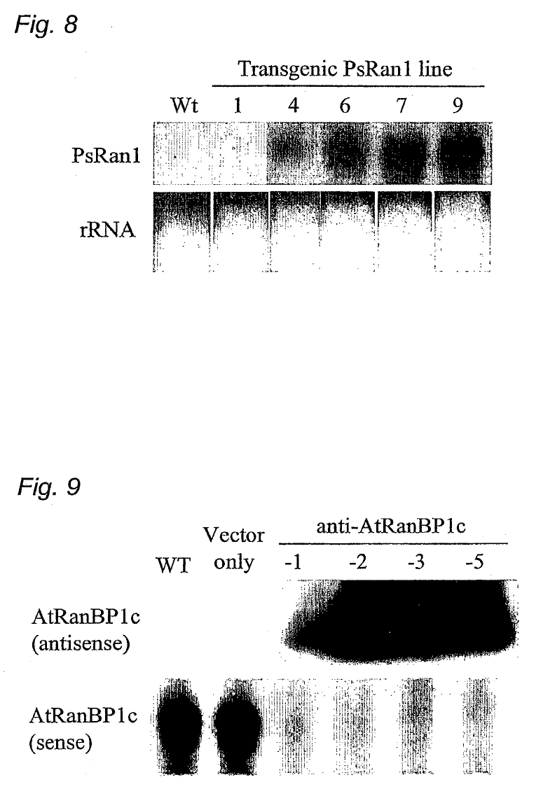

FIG. 8 shows a photograph of Northern blotting analysis of the plants transformed with pLBJ21/PsRan, and FIG. 9 shows a photograph of Northern blotting analysis for the plants transformed with pLBJ21/antisense AtRanBP1c. As shown in FIGS. 8 and 9, transgenes were overexpressed in the transgenic Arabidopsis plants. In addition, it was verified that antisense AtRanBP1c was expressed, and the expression of endogenous AtRanBP1c was inhibited in the corresponding transgenic plants, as shown in FIG. 9.

Experiment 2. The Phenotypes of 5 Different Transgenic Arabidopsis Lines

(i) The phenotypes of a Root Length

The length of Arabidopsis roots according to Examples 1 to 5 was measured, and the results were shown in Tables 1 and 2.

TABLE 1

| | Mean root length (mm) |
|---|---|
| Wild type | 5 ± 1.5 |
| pLBJ21/PsRan-1 | 6 ± 1.8 |
| pLBJ21/PsRan-4 | 15 ± 4 |
| pLBJ21/PsRan-7 | 13 ± 3.6 |

TABLE 2

| | Mean root length (mm) |
|---|---|
| Wild type | 7.5 ± 1.5 |
| pLBJ21/antisense AtRanBP1c-1 | 12 ± 2.3 |
| pLBJ21/antisense AtRanBP1c-2 | 20 ± 4.1 |
| pLBJ21/antisense AtRanBP1c-3 | 18 ± 4.3 |
| pLBJ21/antisense AtRanBP1c-5 | 9 ± 1.2 |

As shown in FIG. 8, Table 1, and Table 2, the length of roots of the transgenic Arabidopsis (pLBJ21/PsRan-4) and the transgenic Arabidopsis (pLBJ21/PsRan-7) overexpressing PsRan were greater than that of the wild type plants or the transgenic Arabidopsis (pLBJ21/PsRan-1) that do not overexpress the PsRan gene. In addition, the root of the Arabidopsis transformed with pLBJ21/antisense AtRanBP1c was also longer than that of wild type plants.

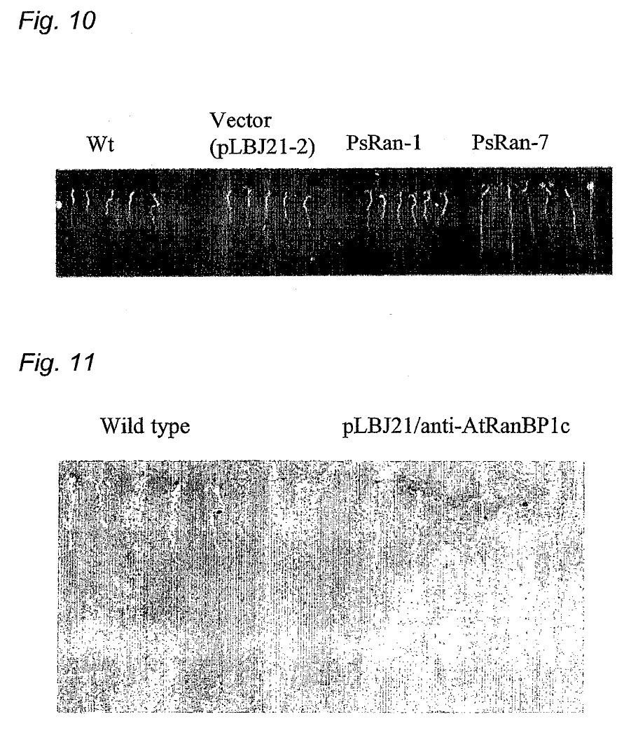

FIG. 10 shows a photograph of roots of Arabidopsis transformed with pLBJ21/PsRan-7, and other control plants. The length of transgenic pLBJ21/PsRan-7 roots is greater than that of wild type or the Arabidopsis transformed with pLBJ21.

FIG. 11 shows a photograph of a root length of Arabidopsis transformed with pLBJ21/antisense AtRanBP1c. It is clear that a root length of Arabidopsis transformed with pLBJ21/antisense AtRanBP1c is greater than that of wild type.

For anatomical analysis of 5 different transgenic Arabidopsis lines of the above Examples, root tissues were fixed with Navishins solution (Mauseth, J. D., Montenegro, G., and Walckowiak, A. M. (1984) Can. J. Bor. 62, 847-857). After the fixation, the plants tissues were dried through the standardized ethanol step. The ethanol was exchanged with xylene before being introduced into paraffin, and then the tissues were cut to 7 μn and were stained to observe with a standard bright microscope. Each root was measured every 24 hours using a microscope of 40× magnification for anatomic use, and a ruler. The mean apical growth rate was calculated from the growth means of transgenic plants or wild-type plants. A statistical analysis was conducted using a SAS program, and the measurement was conducted with micrometer scale rulers in the objective lens of 6.3× magnification. A wild-type Arabidopsis was analyzed for comparison.

The following Table 3 presents the microscopic anatomical difference between one of the transgenic plants, pLBJ21/antisense AtRanBP1c, and wild types.

TABLE 3

| | Mean root length (mm) | % wild type |
|---|---|---|
| Example 5 | 9.0 | 164 |

Mean root length of the transgenic plant transformed with pLBJ21/antisense AtRanBP1c in the above Table 3 was approximately 1.6 times longer than that of the wild type. This long root phenotype is due to the increase in mean length of the epidermis, cortex and endodermis, as can be seen in Table 4.

TABLE 4

| | Mean cell length (μm) | | Mean cell width (μm) | |
|---|---|---|---|---|
| | Example 5 | | | |
| | (pLBJ21/ | % wild | Example 5 | % wild |
| | antisense | type | (pLBJ21/antisense | type |
| Cell tissues | AtRanBP1c) | plants | AtRanBP1c) | plants |
| Epidermis | 157.0 | 173 | 14.8 | 103 |
| Cortex | 146.0 | 151 | 28.0 | 116 |
| Endodermis | 107.0 | 130 | 15.9 | 98 |

TABLE 5

| | The growth of root apical cells | |
|---|---|---|
| | Increased length (μm)/day | % wild type Example |
| Wild type | 505 | 100 |
| Example 5 | 750 | 149 |

In addition, as seen in Table 5, the growth rates of root apical cells of transgenic plants are approximately 1.5 times that of wild types.

(ii) The Measurement of Sensitivity to IAA of Transgenic Plants

In order to examine the response to IAA (indole acetic acid) of transgenic plants, seeds of the transgenic Arabidopsis (pLBJ21/antisense AtRanBP1c) were surface-sterilized with a common bleaching solution for 15 minutes. They were then washed with sterile water, and then dried in the air. Plates for germination media (GM-sucrose) were prepared and seeds were placed therein, and incubated at 4° C. for 72 hours so that germinations would occur simultaneously. Various concentrations of IAA's were added to the plates and the media were transferred to growth chambers and grown under all same conditions. Seeds were germinated and grown for 10 days (Modified from Knee and Hangarter, 1996). After 10 days, the lengths and shapes of roots were measured and observed. Those of the transgenic plants of Examples were compared with those of wild-type plants, and the results are presented in the following Table 6.

TABLE 6

| | Mean root length | Mean root length of wild type; |
|---|---|---|
| Auxin | of Example 5 ± | Average root length of vector |
| (M) | standard deviation (mm) | controls (mm) |
| 0 | 19.5 ± 0.9 | 9.6; 7.3 |
| 10⁻¹⁰ | 7.5 ± 3.9 | 9.1; 10.8 |
| 10⁻¹¹ | 8.2 ± 1.8 | 6.5; 8.1 |
| 10⁻¹² | 8.7 ± 2.7 | 6.4; 11.8 |
| 10⁻¹³ | 14.5 ± 3.1 | 6.8; 6.7 |
| 10⁻¹⁴ | 13.3 ± 1.9 | 7.6; 11.0 |

In the roots of wild-type plant, the growth is generally promoted by the application of 10⁻¹⁰ M-10⁻⁶ M of auxin, and it is suppressed by a concentration exceeding the above range. As seen in Table 6, the sensitivity of the transgenic plants to auxin has been largely increased. Therefore, the growth of roots was seriously suppressed even by the application of 10⁻¹⁰ M-¹⁰⁻¹² M of auxin. The suppression of root growth was caused by the failure of somatic cell divisions that occurs in the dividing cells of the transgenic plants in Example 5. From this, it is obvious that the growth of primary roots in the transgenic plant of Example 5 was promoted by a very low concentration of endogenous auxin (less than 10⁻¹⁰ -10⁻¹² M).

(iii) Seed Size Comparison of Transgenic Plants

The seeds of the T3 generation of 5 different transgenic lines according to Examples 1 to 5 were harvested and weighed. The results are presented in Table 7.

TABLE 7

| Transgenic plants | Weight/100 seeds | Standard deviation |
|---|---|---|
| Wild type | 1.76 × 10⁻⁵ g | 1.69 × 10⁻⁶ |
| pLBJ21/PsRan-1 | 2.06 × 10⁻⁵ g | 1.67 × 10⁻⁶ |
| pLBJ21/PsRan-2 | 2.08 × 10⁻⁵ g | 8.37 × 10⁻⁷ |
| pLBJ21/PsRan-3 | 2.08 × 10⁻⁵ g | 1.64 × 10⁻⁶ |
| pLBJ21/PsRan-4 | 2.00 × 10⁻⁵ g | 1.87 × 10⁻⁶ |
| pLBJ21/PsRan-5 | 2.35 × 10⁻⁵ g | 2.16 × 10⁻⁶ |
| pLBJ21/PsRan-6 | 2.24 × 10⁻⁵ g | 1.82 × 10⁻⁶ |
| pLBJ21/antisense | 2.20 × 10⁻⁵ g | 2.74 × 10⁻⁶ |
| AtRanBP1c-1 | | |
| pLBJ21/antisense | 2.22 × 10⁻⁵ g | 2.39 × 10⁻⁶ |
| AtRanBP1c-2 | | |
| pLBJ21/antisense | 2.82 × 10⁻⁵ g | 2.86 × 10⁻⁶ |
| AtRanBP1c-3 | | |
| pLBJ21/antisense | 2.72 × 10⁻⁵ g | 8.37 × 10⁻⁷ |
| AtRanBP1c-5 | | |
| pLBJ21/antisense | 3.48 × 10⁻⁵ g | 2.23 × 10⁻⁶ |
| AtRanBP1b-4 | | |
| pLBJ21/antisense | 3.71 × 10⁻⁵ g | 2.04 × 10⁻⁶ |
| AtRanBP1b-5 | | |

As seen in Table 7, the transgenic plants of the present invention produce seeds with a weight increased by 1.2 to 2 times or more than the seeds of the wild types. In particular, the Arabidopsis transformed with pLBJ21/antisense AtRanBP1b produced seeds weighting over 2 times that of the wild types. In addition, the transgenic plants of the present invention showed increases in leaf area, stem thickness, and the number of seeds per plant.

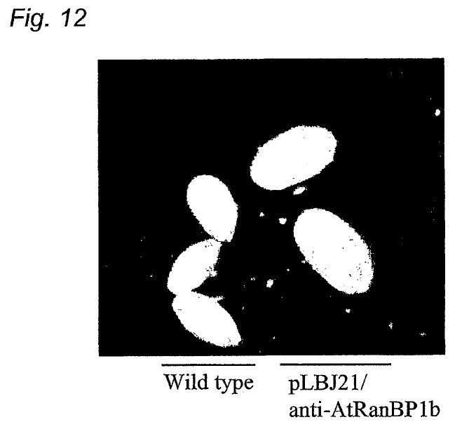

FIG. 12 is a photograph showing that antisense AtRanBP1b/pLBJ21 plants produce bigger seeds than wild types.

(vi) Comparison of the Leaf Area, Hypocotyls Length and Dry Weight of the Transgenic Plants

An Arabidopsis transformed with pLBJ21/antisense AtRanBp1b was germinated in MS medium, the characteristics of Arabidopsis in a 4-leaf-stage were observed 2 weeks after the germination. The results of these experiments are presented in Table 8.

TABLE 8

| | pLBJ21/antisense | |
|---|---|---|
| | AtRanBP1b | Wild type |
| Dry weight (μg) | 8.0 (±1.39) | 3.5 (±0.22) |
| The length of leaf (mm) | 4.44 (±0.13) | 2.10 (±0.05) |
| The area of leaf (mm²) | 1.96 (±0.07) | 1.4 (±0.03) |
| The length of hypocotyl (mm) | 4.95 (±0.06) | 4.15 (±0.12) |

addition, we measured the yield of seed production, the size of flower, and the length of internode. Plants were grown in soil mixed with humus and vermiculite in the ratio of 50:50 for 7 to 9 weeks in a growth chamber under growth conditions at 23° C., 60 % of humidity, and 12 hours of dark and 12 hours of lights. A liquid fertilizer containing 5% nitrogen, 10% aqueous phosphoric acid, and 5% aqueous potassium was provided to the plants twice a week. The characteristics of the plants were measured and the results are presented in Table 9.

TABLE 9

| | pLBJ21/antisense | |
|---|---|---|
| | AtRanBP1b | Wild Type |
| The number of seeds per silique | 30.24 (±1.97) | 28.43 (±1.5) |
| The number of silique per plant | 63.2 (±4.3) | 38.4 (±3.8) |
| The flower size compared to wild | 156 (±5.4) | 100 |
| type, minor axis (%) | | |
| The flower size compared to wild | 170 (±6.5) | 100 |
| type, major axis (%) | | |
| Diameter of 1^{st} internode (mm) | 0.57 (±0.03) | 0.26 (±0.03) |
| Length of 3^{rd} internode (cm) | 1.22 (±0.1) | 0.87 (±0.08) |

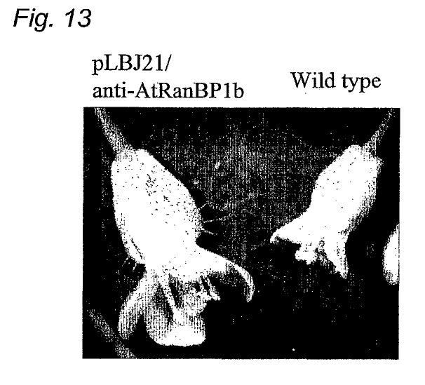

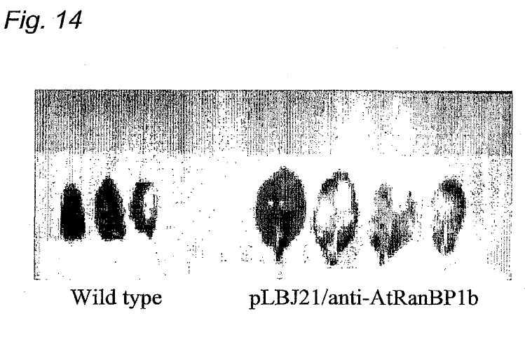

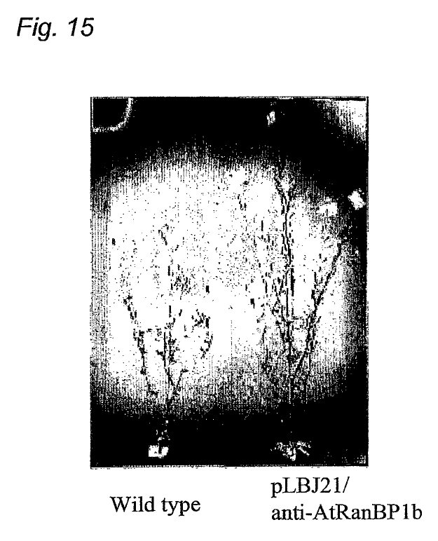

As show in FIGS. 13, 14 and 15, the flower size, the leaf size, and the plant size of Arabidopsis transformed with pLBJ21/antisense AtRanBP1 b were bigger than that of wild type.

Experiment 3. The Phenotype of a Transgenic Tomato

Among the transgenic tomatoes according to Examples 6 to 10, the characteristics of transgenic tomatoes transformed with pLBJ21/AtRanBP1b are presented in Table 10.

TABLE 10

| | pLBJ21/ | |
|---|---|---|
| | AtRanBP1b | Wild type |
| Height of plant (cm) | 180 ± 6.3 | 110 ± 4.8 |
| Diameter of stem (mm) | 11.42 ± 1.3 | 7.6 ± 1.2 |
| Major axis of leaf (mm) | 116 ± 5.3 | 85 ± 3.8 |
| Dry weight of 2 cm by 2 cm leaf | 110 ± 5 | 62 ± 1 |
| sample (mg) | | |
| The thickness of leaf (mm) | 0.58 ± 0.02 | 0.27 ± 0.02 |
| The diameter of immature fruit (mm) | 39 ± 2.6 | 30 ± 1.8 |
| The content of chlorophil per 1 g leaf | 0.58 ± 0.08 | 0.42 ± 0.07 |
| (mg/ml solvent) | | |

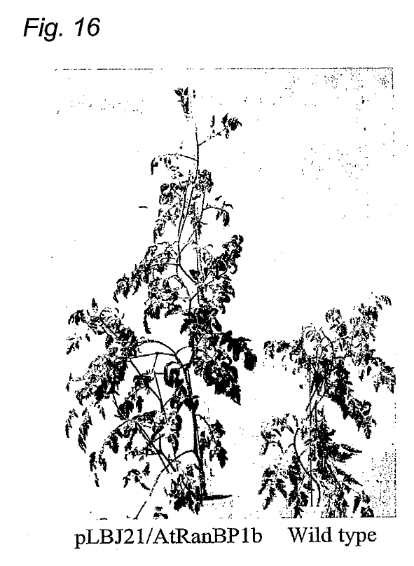

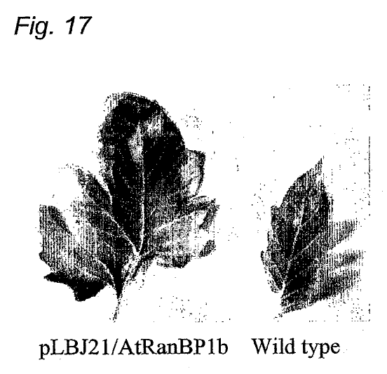

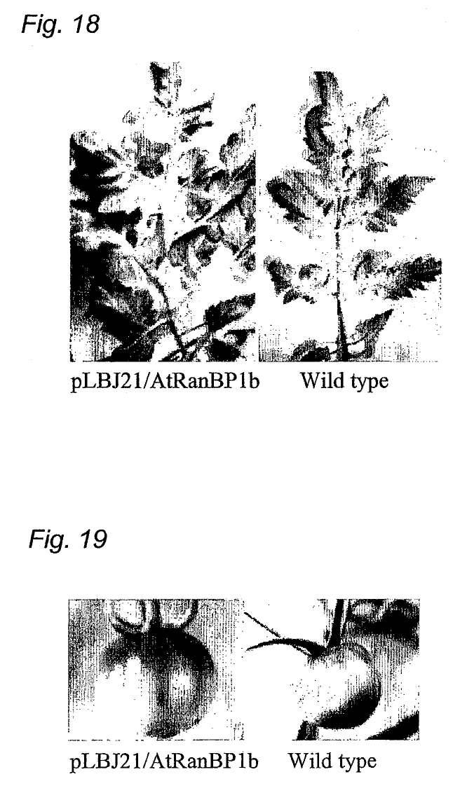

As shown in Table 10, the biomass properties of transgenic tomatoes transformed with pLBJ21/AtRanBP1b, such as plant height, the thickness, weight and size of leaf, the content of chlorophyll per weight and diameter of stem, were all significantly increased. In addition, the diameter of immature fruit increased, because photosynthetic capacity of leaf was increased. As shown in FIGS. 16, 17, 18 and 19, the size of adult body, the size of adult leaf, the stem thickness, and the size of immature fruit of the transgenic tomatoes were bigger than the comparable wild type plants.

Transgenic plants with increased body or seed size or increased root length can be obtained by overexpressing Ran and RanBPs or by suppressing their expressions. Crops transformed with the five kinds of recombinant vectors of the present invention can increase their yield, thus can be applied for development of high yield, super-productive species.

What is claimed is:

1. A PaRan sequence of SEQ.ID.No. 1 isolated from Pissum Satinum.

2. An AtRanBP1b sequence of SEQ.ID.No. 2 isolated from Arabidopsis.

3. An AtRanBP1c sequence of SEQ.ID.No. 3 isolated from Arabidopsis.

4. An antisense AtRanBP1b sequence of SEQ.ID.No. 4, which suppresses the expression of AtRanBP1b genes.

5. An antisense AtRanBP1c sequence of SEQ.ID.No. 5, which suppresses the expression of AtRanBP1c genes.

6. A recombinant vector comprising an antisense AtRanBP1b sequence of SEQ.ID.No. 4 or a part of antisense AtRanBP1b sequence.

7. The recombinant vector according to claim 6 wherein the recombinant vector is pLBJ21/antisense AtRanBP1b( KCTC0850BP).

8. A recombinant vector comprising an antisense AtRanBP1c sequence of SEQ.ID.No. 5 or a part of antisense AtRanBP1c sequence.

9. The recombinant vector according to claim 8 wherein the recombinant vector is pLBJ21/antisense AtRanBP1c( KCTC0851 BP).

10. A recombinant vector comprising genes selected from a group consisting of Ran, AtRanBP1a, AtRanBP1b, AtRanBP1c, RanGAP, RanBPM, RCC1 and RanBP1.

11. The recombinant vector according to claim 10 wherein the recombinant vector is selected from pLBJ21/PsRan (KCTC 0837BP), pLBJ21/AtRanBP1b (KCTC 0838BP) and pLBJ21/AtRanBP1c (KCTC 0839BP).

12. A recombinant plant transformed with a transgenic vector comprising a sense or an antisense sequence of genes involved in the Ran-mediated cellular processes.

13. The transgenic plant according to claim 12 wherein the gene is selected from a group consisting of Ran, AtRanBP1a, AtRanBP1b, AtRanBP1c, RanGAP, RanBPM, RCC1, and RanBP1.

14. The transgenic plant according to claim 12 wherein the recombinant vector is the recombinant vector of claim 6, the recombinant vector of claim 8 or recombinant vector of claim 10.

15. The transgenic plant according to claim 12 wherein the transgenic plant is selected from a group consisting of a plant transformed with pLBJ21/antisense AtRanBP1b (KCTC0850BP), pLBJ21/antisense AtRanBP1c (KCTC0851BP), pLBJ21/PsRan (KCTC 0837BP), pLBJ21/AtRanBP1b (KCTC 0838BP), and pLBJ21/AtRanBP1c (KCTC0839BP).

16. A method for generating transgenic plants comprising the step of transforming a recombinant vector containing the genes involved in Ran-mediated cellular processes into the plants, or knocking out the genes.

17. The method according to claim 16 wherein the recombinant vector comprises a promoter that can be either express a sense or an antisense sequence of genes or sense RNA and antisense RNA in the same times.

18. The method according to claim 16 wherein the genes are selected from a group consisting of Ran, AtRanBP1a, AtRanBP1b, AtRanBP1c, RanGAP, RanBPM, RCC1 and RanBP1.

19. The method according to claim 18 wherein the Ran is a protein that is 70% or more identical with amino acid.

20. The method according to claim 18 wherein the AtRanBP1b or AtRanBP1c is a protein that is 50% or more identical with amino acid.

21. The method according to claim 18 wherein the AtRanBP1a, RanGAP, RanBPM, RCC1 or RanBP1 is a protein that is 35% or more identical with amino acid.

22. The method according to claim 16 wherein the genes comprise Ran-binding domain.

23. The method according to claim 16 wherein the antisense sequence is a whole or a part of antisense sequence.

24. The method according to claim 16 wherein the knocking-out method is a method selected from a group consisting of transposon, UV-treatment, and EMS-treatment.
